(19) United States
(12) Patent Application Publication
(10) Pub. No.: US 2025/0042860 A1
(43) Pub. Date: Feb. 6, 2025

(54) Process for Chemical Synthesis of Ergothioneine and Methods of Use

(71) Applicant: NANJING NUTRABUILDING BIO-TECH CO., LTD., Nanjing (CN)

(72) Inventors: Yongxiao ZHU, Nanjing (CN); Dongdong YU, Nanjing (CN); Ying ZHU, Nanjing (CN); Xi ZHU, Nanjing (CN); Kylin LIAO, Nanjing (CN)

(21) Appl. No.: 18/921,672

(22) Filed: Oct. 21, 2024

Related U.S. Application Data
(63) Continuation of application No. PCT/CN2022/088496, filed on Apr. 22, 2022.

(51) Int. Cl. C07D 233/84

(52) U.S. Cl. CPC C07D 233/84

(57) ABSTRACT

The present invention provides a process for chemical synthesis of different optical forms of ergothioneine (e.g., L-form or D-form or any mixture thereof). Another aspect of the invention relates to a composition comprising a non-racemic mixture of L-ergothioneine and D-ergothioneine.

### CROSS REFERENCE TO RELATED APPLICATION

This application is a continuation application of International Patent Application No. PCT/CN2022/088496, filed on Apr. 22, 2022, the contents of which are incorporated herein by reference in their entirety.

### BACKGROUND OF THE INVENTION

Ergothioneine (EGT) was discovered by Charles Tanret in 1909, whilst investigating the ergot fungus, Claviceps purpurea. It is a naturally occurring, sulfur-containing amino acid, and is known to be synthesized only by non-yeast fungi, and certain bacteria. Yet despite the inability to be synthesized by human being, ergothioneine is found in the entire human body, with the highest levels found in kidneys, liver, red blood cells, and semen. While a certain function still needs to be clarified, ergothioneine may be important to human health due to the prevalence of a special transporter in many tissues (Journal of Functional Foods, Volume 77, February 2021, 104326). Studies have highlighted its antioxidant abilities in vitro, and possible cytoprotective capabilities in vivo (Scientific Reports volume 8, Article number: 1601 (2018)). Since human cannot synthesize ergothioneine, it can only be acquired through diet. Studies in animals and humans have found no toxicity or adverse effects to be associated with ergothioneine administration, even at high doses, and recently, ergothioneine (Tetrahedron, Paris, France) has attained European Food Safety Authority approval in the European Union and is generally recognized as a safe supplement by the Food and Drug Administration in the US (GRAS notice No. 734) (FEBS Letters 592 (2018) 3357-3366).

Thus, ergothioneine is a potentially useful dietary supplement. Present methods for preparation of ergothioneine includes extraction from natural sources, biosynthesis pathway and chemical synthesis, for example, Chinese patent CN 106831597 B discloses a method for preparing ergothioneine from mushroom; U.S. Pat. Nos. 10,544,437 B2 and 10,167,490 B2 disclose methods for ergothioneine biosynthesis; and U.S. Pat. Nos. 5,438,151 A; 7,767,826 B2; 8,399,500 B2; 9,908,854 B2; and 9,428,463 B1 disclose methods for chemical synthesis of ergothioneine.

The present invention provides a process for chemical synthesis of ergothioneine. Interestingly, the non-natural enantiomers D-ergothioneine can also be easily obtained from D-histidine by using the present method. Hence, each component of the composition can be either a pure optical isomer (e.g., L-form or D-form), or a mixture of both isomers depending on the choice of the starting compounds, which can be present in pure L- or D-form, or as mixtures thereof. Moreover, this present process greatly reduces the total number of synthetic steps for preparing EGT, and improves the overall yield, thereby lowering the cost. Other than that, this process disclosed in this application can also be economically and conveniently used for industrial production of ergothioneine.

### BRIEF SUMMARY OF THE INVENTION

This summary is provided to introduce a selection of concepts in a simplified form that are further described below in the Detailed Description. This summary is not intended to identify key features or essential features of the claimed subject matter, nor is it intended to be used to limit the scope of the claimed subject matter.

One aspect of the invention provides a novel process for chemical synthesis of different optical isomers of ergothioneine, or a physiologically acceptable salt thereof, comprising the following successive steps:
(a) in a first solvent, reacting histidine (compound I) with a protective reagent I and a protective agent II in the presence of a base, thereby obtaining a compound (II); wherein the histidine is L-form, D-form, or a mixture of L- and D-forms in any ratio;
(b) in a second solvent, reacting the compound (II) with an alkylation reagent, thereby obtaining a compound (III);
(c) in a third solvent, hydrolyzing the compound (III) in the presence of an acid, obtaining a compound (IV);
(d) in a fourth solvent, reacting the compound (IV) with a halogenated reagent, and then reacting with a vulcanization reagent selected from the group consisting of L-cysteine, (D,L)-cysteine, and D-cysteine, and then reacting with a cleavage reagent, thereby obtaining ergothioneine (compound V) after a post-treatment process;
wherein the obtained ergothioneine is in L-form, D-form, or a mixture of L- and D-form in any ratio.

In some embodiments, the compound (II) has the following structure:

Examples of R include

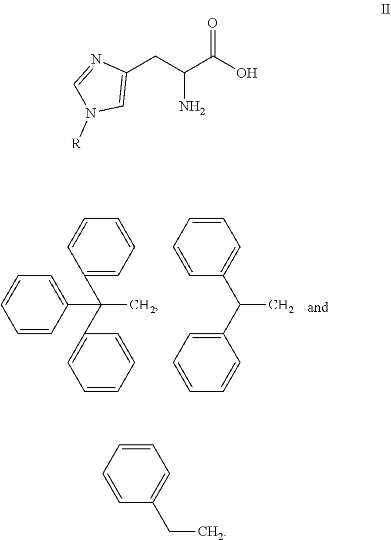

In some embodiments, the compound (III) has the following structure:

Examples of R include

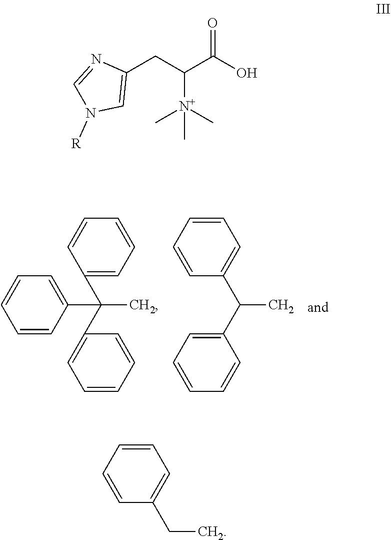

In some embodiments, the compound (IV) has the following structure:

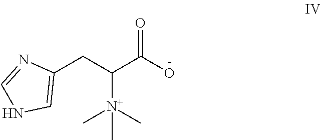

In some embodiments, the compound (V) has the following structure:

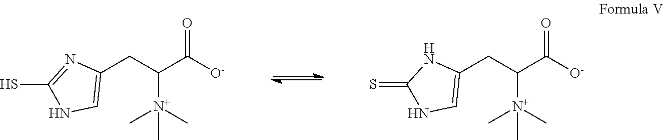

In some embodiments, in step (a),
the protective reagent 1 is selected from the group consisting of dimethyldichlorosilane, trimethylchlorosilane, and dichlorodiphenylsilane;
the protective reagent 2 is selected from the group consisting of triphenylchloromethane, chlorodiphenylmethane, and benzyl bromide;
the first solvent is selected from the group consisting of methylene dichloride, tetrahydrofuran and trichloromethane; and
the base is selected from the group consisting of pyridine, N,N-diisopropylethylamine and triethylamine.

In some preferred embodiments, in step (a), the protective reagent 1 is dimethyldichlorosilane, the protective reagent 2 is triphenylchloromethane, the first solvent is dichloromethane, and the base is triethylamine or pyridine.

In some embodiments, in step (a), the molar ratio of the base and the compound (I) ranges from 1:1 to 3:1; molar ratio of the protective reagent 1 and the compound (I) ranges from 1:1 to 3:1; molar ratio of the protective reagent 2 and the compound (I) ranges from 1:1 to 3:1; and the reaction temperature ranges from 0-80° C., more preferably, 10-40° C.

In some embodiments, in step (b), the alkylation reagent is selected from the group consisting of dimethyl sulfate, methyl iodide, methyl bromide or methyl chloride; and the second solvent is selected from the group consisting of acetonitrile, methanol, ethanol or water.

Preferably, in step (b), the alkylation reagent is dimethyl sulfate, and the second solvent is methanol.

In some embodiments, in step (b), the molar ratio of the alkylation reagent and the compound (II) ranges from 1:1 to 3:1; and the reaction temperature ranges from 20 to 100° C., more preferably, 20-30° C.

In some embodiments, in step (c), the third solvent is selected from the group consisting of acetonitrile, methanol, ethanol or water; the acid is selected from the group consisting of hydrochloric acid, sulfuric acid, trifluoroacetic acid, or trifluoromethanesulfonic acid.

Preferably, in step (c), the third solvent is water, and the acid is hydrochloric acid.

In some embodiments, in step (c), molar ratio of the acid and compound (III) ranges from 1:1 to 10:1, preferably from 2:1 to 5:1; and the reaction temperature ranges from 20 to 80° C., or preferably 70-80° C.

In some embodiments, in step (d), the fourth solvent is selected from the group consisting of water, methanol and ethanol; the halogenated reagent is selected from the group consisting of bromine, dibromohydantoin, imidazolidinedione, bromosuccinimide, iodosuccinimide, and chlorosuccinimide; and the cleavage reagent is selected from the group consisting of cysteamine, sodium thiosulfate, ammonium thiocyanate and mercaptopropionic acid.

Preferably, in step (d), the fourth solvent is water, the halogenated reagent is bromosuccinimide, and the cleavage reagent is sodium thiosulfate.

In some embodiments, in step (d), the molar ratio of the vulcanization reagent and the compound (IV) ranges from 1:1 to 10:1, or preferably from 3:1 to 5:1.

In some embodiments, in step (d), the molar ratio of the cleavage reagent and compound (IV) ranges from 1 to 5:1, preferably from 2:1 to 3:1; and the reaction temperature ranges from 0 to 100° C., or preferably 70 to 90° C.

In some embodiments, in step (d), the post-treatment process comprises at least one step selected from the group consisting of filtration, decolorization, electrodialysis, concentration, ion-exchange chromatography and recrystallization in a recrystallization reagent.

Preferably, in step (d), the post-treatment process is recrystallization.

Examples of the recrystallization reagent includes isopropanol, ethanol, methanol, water, or any combination or mixture thereof.

Another aspect of the invention provides an antioxidant composition, comprising a mixture of L-ergothioneine and D-ergothioneine.

In some embodiments, the mixture comprises less than or equal to 100% (e.g., 30-80% or 45-55%) by enantiomeric equivalents of the L-ergothioneine and greater than or equal to 0% (e.g., 20-70% or 45-55%) from by enantiomeric equivalents of the D-ergothioneine.

In some embodiments, the composition is prepared in a form of nutritional, drinking, cosmetic or pharmaceutical composition, for use in a food, drink, nutritional, cosmetic or pharmaceutical products.

In some embodiments, the composition is administrated in a form of capsule, tablet, powder, suspension, solutions, drops, granules, liquids, syrups, functionalized foods, beverages, toothpastes, sublingual articles, food product, food additive, candy, sucker, pastille, food supplement, and suppository.

### DETAILED DESCRIPTION OF THE INVENTION

Reference will now be made in detail to the preferred embodiments of the invention, examples of which are further illustrated. While the invention will be described in conjunction with the preferred embodiments, it will be understood that they are not intended to limit the invention to these embodiments. On the contrary, the invention is intended to cover alternatives, modifications, and equivalents, which may be included within the spirit and scope of the invention as defined by the claims. Furthermore, in the detailed description of the present invention, numerous specific details are set forth in order to provide a thorough understanding of the present invention. However, it will be obvious to one of ordinary skill in the art that the present invention may be practiced without these specific details. In other instances, well-known methods, procedures, components, and other features have not been described in detail as not to unnecessarily obscure aspects of the present invention.

One aspect of the present invention is directed to a novel process for chemical synthesis of ergothioneine, resulting in high product yield without any racemized product. This process can produce ergothioneine either in a pure optical form (e.g., L-form or D-form), or a mixture of both forms in any ratio as needed. Surprisingly, it can directly provide ergothioneine of high-quality standard, thus obviating the need for additional steps of purification. The ergothioneine can be obtained by the following route, depicted in reaction scheme as follows:

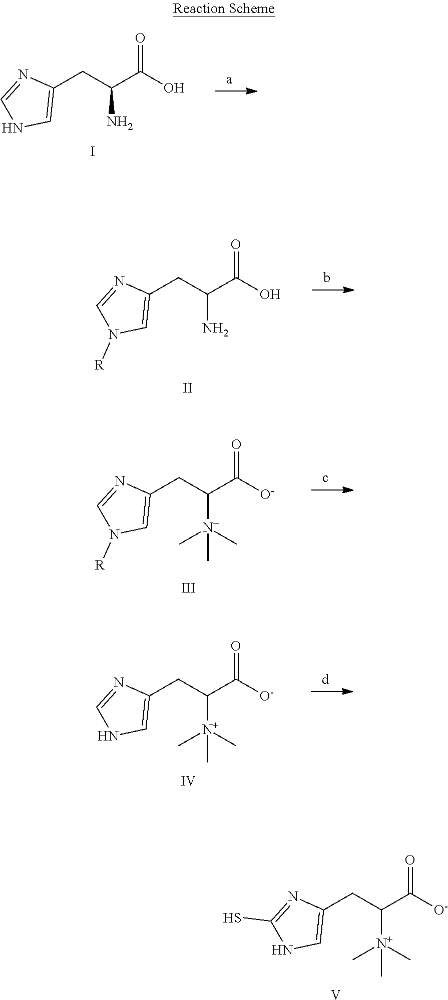

As shown in the scheme above, it includes four successive steps:
(a) in a first solvent, reacting histidine (compound I) with a protective reagent I and a protective agent II in the presence of a base, thereby obtaining a compound (II), wherein the histidine is either in a pure optical form (e.g., L-form or D-form), or a mixture of both forms;
(b) in a second solvent, reacting the compound (II) with an alkylation reagent, thereby obtaining a compound (III);
(c) in a third solvent, hydrolyzing the compound (III) in the presence of an acid, obtaining a compound (IV);
(d) in a fourth solvent, reacting the compound (IV) with a halogenated reagent, and then reacting with a vulcanization reagent selected from the group consisting of L-cysteine, (DL)-cysteine, and D-cysteine, and then reacting with a cleavage reagent thereby obtaining ergothioneine (compound V).

Another aspect of the invention is a composition comprising a mixture of L-ergothioneine and D-ergothioneine, or a physiologically acceptable salt thereof, which can be prepared using the chemical synthesis method according to the present invention. This composition possesses broad applications in treating disease, cosmetic application, or nutritional supplement.

### Definitions

As used herein, the term “or” is meant to include both “and” and “or”. In other words, the term “or” may also be replaced with “and/or.”

As used herein, the singular forms “a,” “an” and “the” are intended to include the plural forms as well, unless the context clearly indicates otherwise.

As used herein, the term “at least” followed by a number is used to denote the start of a range beginning with that number.

As used herein, the term “physiologically acceptable” is taken to designate what is generally safe, non-toxic and neither biologically nor otherwise undesirable and which is acceptable for pharmaceutical, cosmetic or food (human or animal) use, in particular food. “Physiologically acceptable salts” of a compound is taken to designate salts that are physiologically acceptable, as defined above, and which have the desired activity (pharmacological, cosmetic or food) of the parent compound.

As used herein, the term “tautomer” or “tautomeric form” refers to structural isomers of different energies which are interconvertible via a low energy barrier. The different tautomer of a compound is generally interconvertible and present in equilibrium, in solution, in proportions that can vary according to the solvent used, the temperature, or even the pH. Within the framework of the invention, ergothioneine obtained from the present invention can be present as a tautomer between its thiol and thione forms.

Structure of L-ergothioneine (thione-thiol tarutomers) is shown below. It exists predominantly in the thione form at physiological pH.

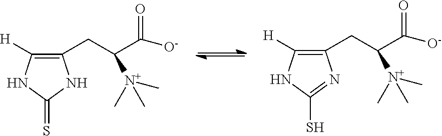

As used herein, the term “optical isomer” or “optical form” refers to any of the various stereo isomeric configurations which may exist for a given compound of the present invention and includes geometric isomers. For example, naturally occurring alanine is the right-hand structure, and the way the groups are arranged around the central carbon atom is known as an L-configuration. The other configuration is known as D-configuration.

As used herein, the term “enantiomers” refers to stereo isomers that are mirror images of each other, but not superimposable. A mixture containing equal quantities of two individual enantiomer forms of opposite chirality is designated as “racemic mixture”.

### EXAMPLES

Synthetic methodologies illustrated herein are intended to exemplify the applicable chemistry through the use of specific examples and are not indicative of the scope of the disclosure.

All reagents used in the Examples were obtained from commercial suppliers without further purification.

### Example 1. Screening of Reaction Conditions for a Better Yield of Desired Product

Step (a): in a first solvent, reacting histidine (compound I) with a protective reagent I and a protective agent II in the presence of a base, thereby obtaining a compound (II), wherein the histidine is either in a pure optical form (e.g., L-form or D-form), or a mixture of both forms;

To achieve a better yield of the desired product, reaction conditions of step (a), including solvent, protective reagents and base, were screened. The obtained products were analyzed by High Performance Liquid Chromatography (HPLC), and results are shown in Table 1.

The results showed that:
for the solvent, use of dichloromethane, trichloromethane or tetrahydrofuran gave a yield higher than 90%;
for the reagent 1, use of dimethyldichlorosilane, dichlorodiphenylsilane and trimethylchlorosilane gave a yield higher than 95%;
for the reagent 2, use of triphenylchloromethane, diphenylchloromethane and benzyl bromide give a yield higher than 95%, however, use of benzyl chloride only gave a yield of around 20%;
for the base, use of pyridine, N,N-diisopropylethylamine, or triethylamine gave a yield higher than 85%.

TABLE 1
Screening of the Reaction Conditions in Step (a)

| | | | | | | Yield |
|---|---|---|---|---|---|---|
| No. | Solvent | Reagent 1 | Reagent 2 | Base | T (° C.) | (%) |
| 1 | dichloromethane | dimethyldichlorosilance | triphenylchloromethane | triethylamine | 10~40° C. | 93% |
| 2 | dichloromethane | dichlorodiphenylsilane | triphenylchloromethane | triethylamine | 10~40° C. | 92% |
| 3 | dichloromethane | dimethyldichlorosilance | triphenylchloromethane | triethylamine | 10~40° C. | 95% |
| 4 | trichloromethane | dimethyldichlorosilance | triphenylchloromethane | pyridine | 10~40° C. | 91% |
| 5 | tetrahydrofuran | dichlorodiphenylsilane | triphenylchloromethane | pyridine | 10~40° C. | 91% |
| 6 | tetrahydrofuran | dichlorodiphenylsilane | triphenylchloromethane | triethylamine | 10~40° C. | 92% |
| 7 | 1,2-dichloroethane | dichlorodiphenylsilane | triphenylchloromethane | triethylamine | 10~40° C. | 94% |
| 8 | dichloromethane | dichlorodiphenylsilane | triphenylchloromethane | N,N- | 10~40° C. | 95% |
| | | | | diisopropyl | | |
| | | | | ethylamine | | |
| 9 | dichloromethane | dichlorodiphenylsilane | diphenylchloromethane | triethylamine | 10~40° C. | 95% |
| 10 | dichloromethane | dichlorodiphenylsilane | benzyl chloride | triethylamine | 10~40° C. | 20% |
| 11 | dichloromethane | dichlorodiphenylsilane | benzyl bromide | triethylamine | 10~40° C. | 96% |
| 12 | dichloromethane | trimethylchlorosilane | triphenylchloromethane | triethylamine | 10~40° C. | 95% |

Step (b): in a second solvent, reacting the compound (II) with an alkylation reagent, thereby obtaining a compound (III);

For step (b), reaction conditions, including methylation reagents, solvent and reaction temperature were screened for a higher yield of desired product (i.e. Compound III). The yield was analyzed by HPLC, and results are listed in Table 2 below.

Results showed that:
for the alkylation reagent, use of dimethyl sulfate, methyl iodide or methyl chloride, dimethyl carbonate gave a yield higher than 85%, however, there is no reaction when trimethyl phosphate (No. 4) is used;
for the solvent, use of acetonitrile, methanol or water gave a yield higher than 80%; however, there is almost no reaction when ethyl acetate is used;
for the reaction temperature, a temperature between 20˜30° C. can give a higher yield, however, when the reaction temperature dropped to 0-10° C. or raised to 60-70° C., the reaction is very slow.

TABLE 2
Screening of the Reaction Conditions in Step (b)

| | | Alkylation reagent/ | | | |
|---|---|---|---|---|---|
| | | molar equivalent to | | | |
| No. | Solvents | Compound II | T (° C.) | | Yield |
| 1 | methanol | dimethyl sulfate/1.2 | 20~30° | C. | 90% |
| 2 | methanol | iodomethane/1.2 | 20~30° | C. | 90% |
| 3 | methanol | chloromethane/1.2 | 20~30° | C. | 85% |
| 4 | acetonitrile | trimethyl phosphate/ | 20~30° | C. | no |
| | | 1.2 | | | reaction |
| 5 | acetonitrile | dimethyl sulphate/1.2 | 20~30° | C. | 85% |
| 6 | water | dimethyl sulphate/1.2 | 20~30° | C. | 80% |
| 7 | ethanol | dimethyl sulphate/1.2 | 20~30° | C. | 82% |
| 8 | ethyl acetate | dimethyl sulphate/1.2 | 20~30° | C. | no |
| | | | | | reaction |
| 9 | tetrahydrofuran | dimethyl sulphate/1.2 | 0~10° | C. | very slow |
| | | | | | reaction |
| 10 | tetrahydrofuran | dimethyl sulphate/1.2 | 60~70° | C. | very slow |
| | | | | | reaction |
| 11 | water | dimethyl sulphate/1.2 | 20-30° | C. | 55% |
| 12 | methanol | dimethyl carbonate/1.2 | 20-30° | C. | no |
| | | | | | reaction |

Step (c): in a third solvent, hydrolyzing the compound (III) in the presence of an acid, obtaining a compound (IV);

For step (c), the reaction conditions, including the solvent, acid and reaction temperature were screened for a higher yield of desired product. The results were analyzed by HPLC, and results are listed in Table 3 below.

The results showed that:
for the solvent, use of methanol, acetonitrile or water give a yield higher than 80%; however, the reaction is very slow when tetrahydrofuran is used as solvent;
for the acid, use of hydrochloric acid, sulfuric acid, trifluoroacetic acid or trifluoromethanesulfonic acid can give a good yield, especially, hydrochloric acid and sulfuric acid, which can give a yield higher than 95%;
for the temperature, a temperature of 70-80° C. give a better yield than the lower temperature of 50° C.˜60° C.

TABLE 3
Screening of Reaction Conditions in Step (c)

| No. | Solvents | Acid/Equivalent | T (° C.) | Yield |
|---|---|---|---|---|
| 1 | Water | hydrochloric acid/1.5 | 70~80° C. | 98% |
| 2 | methanol | hydrochloric acid/1.5 | 70~80° C. | 95% |
| 3 | acetonitrile | hydrochloric acid/1.5 | 70~80° C. | 90% |
| 4 | tetrahydrofuran | hydrochloric acid/1.5 | 70~80° C. | very slow |
| | | | | reaction |
| 5 | water | hydrochloric acid/1.5 | 50~60° C. | very slow |
| | | | | reaction |
| 6 | methanol | hydrochloric acid/1.5 | 50~60° C. | very slow |
| | | | | reaction |
| 7 | water | sulfuric acid/1.5 | 70~80° C. | 95% |
| 8 | methanol | trifluoroacetic acid/1.5 | 50~60° C. | 92% |
| 9 | methanol | trifluoromethanesulfonic | 50~60° C. | 89% |
| | | acid/1.5 | | |

### Example 2. Process for Chemical Synthesis of Ergothioneine

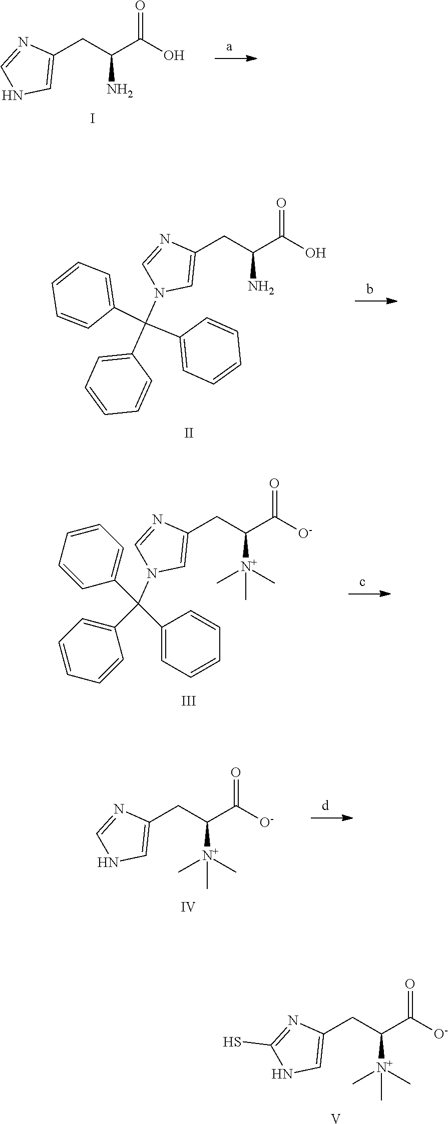

(a) 300.0 g histidine was suspended in 3.0 L dichloromethane under the protection of nitrogen, followed by the addition of 274.5 g dichlorodimethylsilane, and then the solution was cooled to 15° C. Under stirring, 723.90 g triethylamine were added to the solution drop by drop without exceeding 20° C. After the addition of triethylamine, the reaction mixture was heated at reflux temperature for 4 h, and then cooled to 15° C. After cooling, 592.92 g of triphenylchloromethane was dissolved in 1.2 L of dichloromethane and then added to the mixture in a dropwise manner without exceeding 30° C. After the addition of triphenylchloromethane, the reaction mixture was heated under reflux for 2 h, and then cooled to 25° C. Next, 3 L of water was added to the reaction mixture and stirred for an additional 20 min. After stirring, the reaction mixture was filtered and the filtrate was rinsed with 1 L water, followed by 500 ml dichloromethane. The filtrate was then allowed to dry at 60° C. to give compound (II) with a yield of 99%. This product was used in the next step without further purification.

¹H NMR (400 MHZ, CD₃OD-d₆) δ 7.17-7.44 (m, 16H), 6.87 (s, 1H), 3.84 (dd, 1H), 3.17 (dd, 1H), 2.98 (dd, 1H).
(b) 120 g of compound (II) from step (a) was added to 1000 ml methanol, followed by the addition of 145.8 g potassium carbonate, and then the solution was cooled to 30° C. Under stirring, 106.9 g dimethyl sulfate was then added drop by drop. After the addition of dimethyl sulfate, the reaction mixture was heated to 40° C. and maintained for 8 h. Next, the resulting product was filtered, and the methanol left in the filtrate was evaporated off at 40° C. to give a white solid. The obtained white solid was then suspended in water and stirred for 1˜2 h at room temperature. The solid was further filtered and evaporated to dryness at 70˜80° C. to give compound (III) with a yield of 95%. The product was used in the next step without further purification.

¹H NMR (400 MHZ, CD₃OD-d₆) δ 7.16-7.44 (m, 16H), 6.91 (s, 1H), 3.89 (dd, 1H), 3.19-3.28 (m, 11H).
(c) 50 g compound (III) from step (b) was dissolved in 500 ml water, followed by the addition of 11.5 g concentrated hydrochloric acid at 25° C. The reaction mixture was heated to 70-80° C., and maintained for 8 h under stirring. Thin layer chromatography (TLC) was used to monitor the progress of the reaction. At the end of the reaction, the reaction mixture was cooled to 20-30° C. Then, the resulting product was extracted with 500 ml dichloromethane. The dichloromethane extraction was then concentrated. The resulting product was further purified by recrystallization from ethanol to give compound (IV) with a yield of 98%. The product was used in the next step without further purification.

¹H NMR (400 MHZ, D₂O-d₆) δ 7.58 (S, 1H), 6.87 (s, 1H), 3.79 (dd, 1H), 3.09-3.15 (m, 11H).
(d) 15 g compound (IV) from step (c) was dissolved in 150 ml water, followed by the addition of 15.1 g bromosuccinimide. After stirring for 10 min, 46 g L-cysteine was added and stirred for an additional 1 h, followed by addition of 18.8 g sodium thiosulfate. Then, the reaction mixture was heated to 90-100° C., and maintained for 15 h. At the end of the reaction, the reaction mixture was cooled down and filtered. The pH of the aqueous phase retained was adjusted to neutral, and then desalinized. The desalinized mixture was further filtered, and the obtained filtrate was evaporated to dryness at 70-80° C. The resulting product was further purified by recrystallization from the combination of 5 ml water and 75 ml isopropanol to give the desired product with a yield of 80%. The descried product consists of 88% ergothioneine (V).

¹H NMR (400 MHZ, D₂O-d₆) δ 6.72 (S, 1H), 3.83 (dd, 1H), 3.09-3.15 (m, 11H).

### Example 3. Process for Chemical Synthesis of Ergothioneine

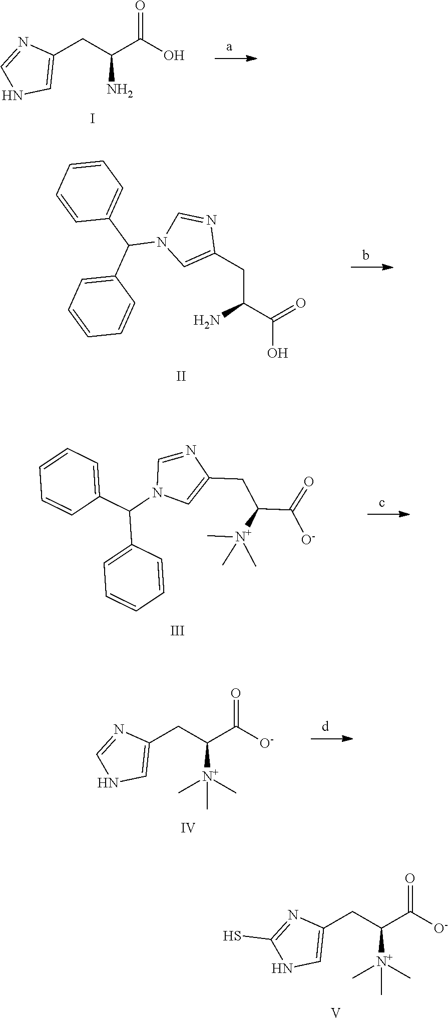

(a) 200.0 g histidine was suspended in 2.0 L dichloromethane, followed by the addition of 183 g dichlorodimethylsilane under the protection of nitrogen, then the solution was cooled to 15° C. Under stirring, 482.6 g triethylamine was added drop by drop without exceeding 20° C. After the addition of triethylamine, the reaction mixture was heated under reflux for 6 h, and then cooled to 15° C. After cooling, 592.92 g of diphenylchloromethane was dissolved in 1.2 L of dichloromethane, and then added to the mixture in a dropwise manner without exceeding 30° C. After the addition of diphenylchloromethane, the reaction mixture was heated under reflux for 4 h, and then cooled to 25° C. After cooling, 2 L water was added and stirred for an additional 20 min. The solid was filtered and rinsed with 1 L water, followed by 400 ml dichloromethane. The solid obtained was then allowed to dry at 60° C., giving the off-white solid of compound (II) with a yield of 94%. The product was used in the next step without further purification.

¹H NMR (400 MHZ, CD₃OD-d₆) δ 7.18-7.42 (m, 6H), 6.87 (s, 1H), 3.84 (dd, 1H), 3.17 (dd, 1H), 2.98 (dd, 1H).
(b) 150 g compound (II) from step (a) was added to 1000 ml methanol, followed by the addition of 182.2 g potassium carbonate, and the solution was cooled to 30° C. Under stirring, 133.6 g dimethyl sulfate was added dropwise. After the addition of dimethyl sulfate, the reaction mixture was heated to 40° C. and maintained for 8 h. After 8 h reaction, the resulting product was filtered and the methanol left in the filtrate was evaporated off at 40° C. to give a white solid. The obtained white solid was then suspended in water, and stirred for 1˜2 h at room temperature. After stirring, the solid was filtered and evaporated to dryness at 70˜80° C. to give the compound (III) with a yield of 85%. The product was used in the next step without further purification.

¹H NMR (400 MHZ, CD₃OD-d₆) δ 7.16-7.44 (m, 11H), 6.91 (s, 1H), 3.89 (dd, 1H), 3.19-3.28 (m, 11H).
(c) 50 g of compound (III) from step (b) was added to 500 ml of water, followed by the addition of 50 g concentrated hydrochloric acid at 25° C. After the addition of hydrochloric acid, the reaction mixture was heated to 70-80° C. and maintained for 12 h with stirring. TLC was used to monitor the progress of the reaction. At the end of the reaction, the reaction mixture was cooled to 20-30° C. Then, the resulting product was extracted with 500 ml dichloromethane. The dichloromethane extraction was concentrated. The resulting product was further purified by recrystallization from ethanol to give the compound (IV) with a yield of 96.3%.

¹H NMR (400 MHZ, D₂O-d₆) δ 7.58 (S, 1H), 6.87 (s, 1H), 3.79 (dd, 1H), 3.09-3.15 (m, 11H).
(d) 15 g compound (IV) from step (c) was added to 150 ml water, followed by the addition of 6.3 g concentrated sulfuric acid and 15.1 g of bromosuccinimide, and the solution was stirred for 10 min. After 10 min stirring, L-cysteine was added and stirred for an additional 1 h, followed by addition of 18.8 g sodium thiosulfate. Then, the reaction mixture was heated to 90-100° C. and maintained for 15 h. At the end of the reaction, the reaction mixture was cooled and filtered. The pH of the aqueous phase retained was adjusted to neutral, and then desalinized. The desalinized mixture was further filtered, and the obtained filtrate was evaporated to dryness at 70-80° C. The resulting product was further purified by recrystallization from the combination of 5 ml of water and 65 ml of isopropanol, giving the desired product with a yield of 75%. The desired product consists of 94% ergothioneine (V).

¹H NMR (400 MHZ, D₂O-d₆) δ 6.72 (S, 1H), δ 6.86 (S, 1H), 3.83 (dd, 1H), 3.09-3.15 (m, 11H).

### Example 4 Process for Chemical Synthesis of Ergothioneine

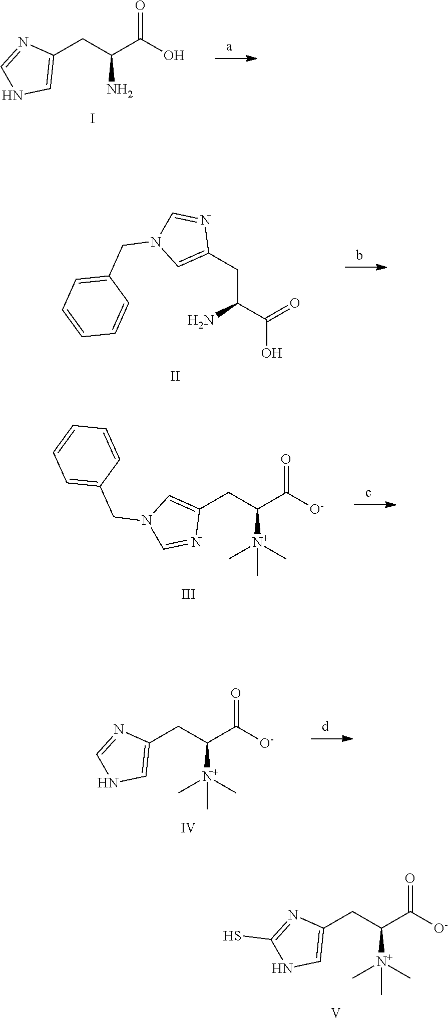

(a) 200.0 g histidine was suspended in 2.0 L dichloromethane, followed by the addition of 183 g dichlorodimethylsilane under the protection of nitrogen, and then the solution was cooled to 15° C. Under stirring, 482.6 g triethylamine was then added drop by drop without exceeding 20° C. After the addition of triethylamine, the reaction mixture was heated under reflux for 6 h, and then cooled to 15° C. After cooling, 242.5 g benzyl bromide was added in 1.2 L dichloromethane dropwise without exceeding 30° C. After the addition of benzyl bromide, the reaction mixture was heated under reflux for 4 h, and then cooled to 25° C. 2 L of water was then added and stirred for an addition of 20 min. After stirring, the resulting product was filtered and then rinsed with 1 L water, followed by 400 mL dichloromethane. The filtrate was allowed to dry at 60° C. to give the off-white solid of compound (II) with a yield of 94%.

¹H NMR (400 MHZ, CD₃OD-d₆) δ 7.18-7.42 (m, 6H), 6.87 (s, 1H), 6.45 (s, 2H), 3.84 (dd, 1H), 3.17 (dd, 1H), 2.98 (dd, 1H).
(b) 100 g compound (II) from step (a) was added to 1000 ml methanol, followed by the addition of 135.2 g potassium carbonate, and the solution was cooled to 30° C. Under stirring, 121.6 g dimethyl sulfate was added dropwise. After the addition of dimethyl sulfate, the reaction mixture was heated to 40° C. and maintained for 8 h. At the end of the reaction, the resulting product was filtered and the methanol left in the filtrate was evaporated off at 40° C. to give the white solid. The white solid was then suspended in water, and stirred at room temperature for 1˜2 h. The solid was further filtrated and heated to dryness at 70˜80° C. to give the compound (III) with a yield of 81%.

¹H NMR (400 MHZ, CD₃OD-d₆) δ 7.12-7.2 (m, 6H), 6.91 (s, 1H), 6.45 (s, 2H), 3.89 (dd, 1H), 3.19-3.28 (m, 11H).
(c) 50 g compound (III) from step (b) was added to 500 ml water, followed by the addition of 25.8 g trifluoroacetic acid at 25° C. The reaction mixture was heated to 70-80° C. and maintained for 12 h with stirring. TLC was used to monitor the progress of the reaction. At the end of the reaction, the mixture was cooled to 20-30° C. Then the resulting product was extracted with dichloromethane. The dichloromethane extraction was then concentrated. The resulting product was further purified by recrystallization from ethanol to give compound (IV) with a yield of 97%.

¹H NMR (400 MHZ, D₂O-d₆) δ 7.58 (S, 1H), 6.87 (s, 1H), 3.79 (dd, 1H), 3.09-3.15 (m, 11H).
(d) 20 g compound (IV) from step (c) was added to 200 ml water, followed by the addition of 20 g bromosuccinimide. After stirring for 0.5 h, L-cysteine was added and stirred for an additional 1 h, followed by addition of 11.3 g ammonium thiocyanate. After pH was adjusted to 12, the reaction mixture was heated to 80-90° C. and maintained for 15 h. At the end of the reaction, the reaction mixture was cooled and filtered. The pH of the aqueous phase retained was adjusted to 6-8, and then the mixture is desalinized. The desalinized mixture was further filtered, and the obtained filtrate was evaporated to dryness at 70-80° C. The resulting product was further purified by recrystallization from the combination of 5 ml of water and 60 ml of isopropanol to give the desired product with a yield of 68%. The desired product consists of 96% ergothioneine (V).

¹H NMR (400 MHZ, D₂O-d₆) δ 6.72 (S, 1H), 3.83 (dd, 1H), 3.09-3.15 (m, 11H).

### Example 5. Process for Chemical Synthesis of Ergothioneine

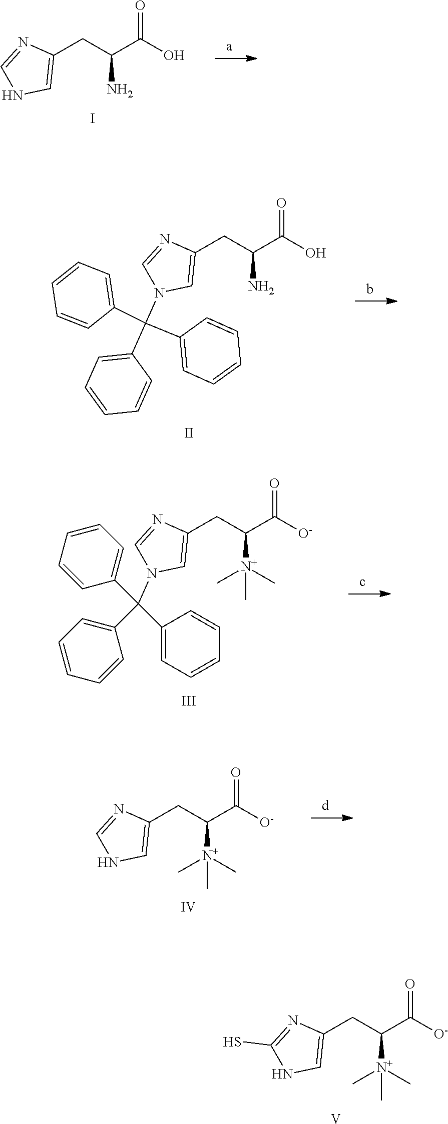

(a) 300.0 g histidine was suspended in 3.0 L dichloromethane, followed by the addition of 274.5 g dichlorodimethylsilane under the protection of nitrogen, and then the solution was cooled to 15° C. Under stirring, 723.90 g triethylamine was added dropwise without exceeding 20° C. After the addition of triethylamine, the reaction temperature was heated under reflux for 4 h, and then cooled to 15° C. After cooling, 592.92 g triphenylchloromethane was added to 1.2 L dichloromethane dropwise without exceeding 30° C. the addition After of triphenylchloromethane, the reaction mixture was heated under reflux for 2 h, and then cooled to 25° C. After cooling, 3 L water was added and stirred for 20 min. After stirring, the resulting product was filtered and then rinsed with 1 L water, followed by 500 ml of dichloromethane. The filtrate was allowed to dry at 60° C. to give the off-white solid compound (II) with a yield of 95%. The product was used in the next step without further purification.

¹H NMR (400 MHZ, CD₃OD-d₆) δ 7.17-7.44 (m, 16H), 6.87 (s, 1H), 3.84 (dd, 1H), 3.17 (dd, 1H), 2.98 (dd, 1H).
(b) 120 g compound (II) was added to 1000 ml methanol, followed by the addition of 145.8 g potassium carbonate, and then the solution was cooled to 30° C. After cooling, 150 g methyl iodide was added dropwise. After the addition, the reaction mixture was heated 40° C. and maintained for 12 h. At the end of the reaction, the reaction mixture was filtered and the methanol left in the filtrate was allowed to dry at 40° C. to give the white solid. The white solid was added in water, and stirred at room temperature for 1˜2 h. The solid was further filtered and allowed to dry at 70˜80° C. to give the compound (III) with a yield of 87%. The product was used in the next step without further purification.

¹H NMR (400 MHZ, CD₃OD-d₆) δ 7.16-7.44 (m, 16H), 6.91 (s, 1H), 3.89 (dd, 1H), 3.19-3.28 (m, 11H).
(c) 50 g compound (III) from step (b) was added to 500 ml water, followed by the addition of 11.5 g concentrated hydrochloric acid at 25° C. After the addition of hydrochloric acid, the reaction mixture was heated to 70-80° C., and maintained for 8 h with stirring. TLC was used to monitor the progress of the reaction. At the end of the reaction, the reaction mixture was cooled to 20-30° C. Then, the resulting product was extracted with 500 ml dichloromethane. The dichloromethane extraction was then concentrated. The resulting product was further purified by recrystallization from ethanol to give the compound (IV) with a yield of 98%. The product was used in the next step without further purification.

¹H NMR (400 MHZ, D₂O-d₆) δ 7.58 (S, 1H), 6.87 (s, 1H), 3.79 (dd, 1H), 3.09-3.15 (m, 11H).
(d) 15 g compound (IV) from step (c) was added to 150 ml of water, followed by the addition of 12 g concentrated hydrochloric acid. After the addition of 7.8 g hydrochloric acid, 10.9 g dibromohydantoin was added and stirred for 20 min. After stirring, L-cysteine was added and stirred for an additional 1 h, followed by the addition of 18.8 g sodium thiosulfate. Then, the reaction mixture was heated to 90-100° C., and maintained for 15 h. At the end of the reaction, the reaction mixture was cooled and filtered. The pH of the aqueous phase retained was adjusted to neutral, and then the mixture was desalinized. The desalinized mixture was further filtered, and the obtained filtrate was evaporated to dryness at 70-80° C. The resulting product was further purified by recrystallization from the combination of 10 ml of water and 50 ml of isopropanol to give the desired product with a yield of 69%. The desired product consists of 99.8% ergothioneine (V).

¹H NMR (400 MHZ, D₂O-d₆) δ 6.72 (S, 1H), 3.83 (dd, 1H), 3.09-3.15 (m, 11H).

### Example 6. Process for Chemical Synthesis of Ergothioneine

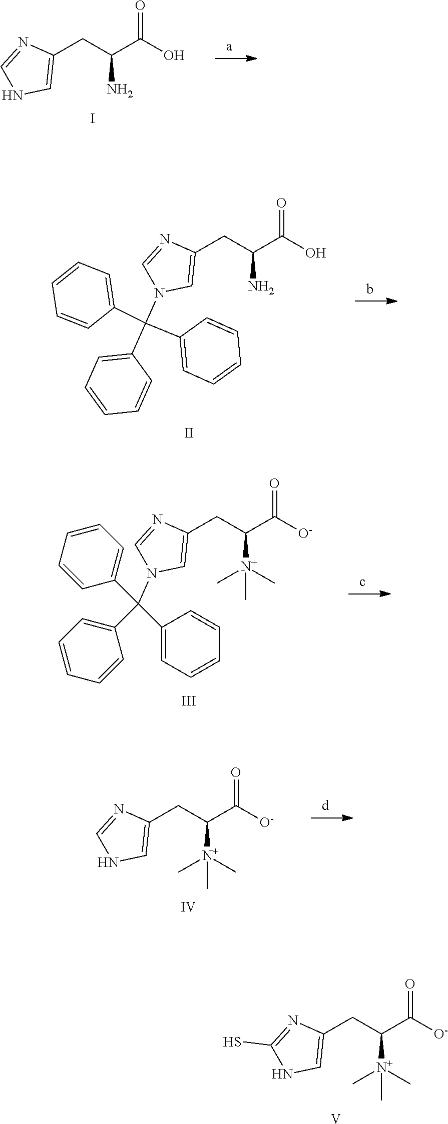

(a) 300.0 g histidine was suspended in 3.0 L dichloromethane, followed by the addition of 274.5 g dichlorodimethylsilane under the protection of nitrogen, and then the solution was cooled to 15° C. After cooling, 723.90 g triethylamine was added dropwise without exceeding 20° C. After the addition of triethylamine, the reaction mixture was heated under reflux for 4 h, and then cooled to 15° C. After cooling, 592.92 g triphenylchloromethane was added to 1.2 L dichloromethane, and then added to the solution dropwise without exceeding 30° C. After the addition, the reaction mixture was heated under reflux for 2 h, and then cooled to 25° C. After cooling, 3 L water was added to the mixture and stirred for 20 min. After 20 min stirring, the producing product was filtered and rinsed with 1 L water, followed by 500 ml dichloromethane. The filtrate was allowed to dry at 60° C. to give the off-white solid of compound (II) with a yield of 95%. The product was used in the next step without further purification.

¹H NMR (400 MHZ, CD₃OD-d₆) δ 7.17-7.44 (m, 16H), 6.87 (s, 1H), 3.84 (dd, 1H), 3.17 (dd, 1H), 2.98 (dd, 1H).
(b) 120 g compound (II) from step (a) was added to 1000 ml methanol, followed by the addition of 145.8 g potassium carbonate, and then the solution was cooled to 30° C. Under stirring, 150 g methyl iodide was added dropwise. After the addition of methyl iodide, the reaction temperature was heated to 40° C. and maintained for 12 h. At the end of the reaction, the producing product was filtered and the methanol left in the filtrate was allowed to dry at 40° C. to give a white solid. The solid was suspended in water and stirred at room temperature for 1˜2 h. The solid was further filtrated and allowed to dry at 70˜80° C. to give the compound (III) with a yield of 87%. The product was used in the next step without further purification.

¹H NMR (400 MHZ, CD₃OD-d₆) δ 7.16-7.44 (m, 16H), 6.91 (s, 1H), 3.89 (dd, 1H), 3.19-3.28 (m, 11H).
(c) 50 g compound (III) was added to 500 ml water, followed by the addition of concentrated hydrochloric acid at 25° C. The reaction mixture was heated to 70-80° C. and maintained for 8 h with stirring. TLC was used to monitor the progress of the reaction. At the end of the reaction, the reaction mixture was cooled to 20-30° C. Then, the resulting product was extracted with 500 ml dichloromethane. The dichloromethane extraction was then concentrated. The resulting product was further purified by recrystallization from ethanol to give the compound (IV) with a yield of 98%. The product was used in the next step without further purification.

¹H NMR (400 MHZ, D₂O-d₆) δ 7.58 (S, 1H), 6.87 (s, 1H), 3.79 (dd, 1H), 3.09-3.15 (m, 11H).
(d) 15 g compound (IV) was added to 150 ml water, followed by the addition of 15.6 g concentrated hydrochloric acid and 10.9 g dibromohydantoin while stirring. After 20 min stirring, D-cysteine was added and stirred for an additional 1 h, followed by addition of 18.8 g sodium thiosulfate. After stirring, the reaction mixture was heated to 90-100° C., and maintained for 15 h. At the end of the reaction, the reaction mixture was cooled and filtered. pH of the aqueous phase retained was adjusted to neutral, and then the mixture was desalinized. The desalinized mixture was further filtered, and the obtained filtrate was evaporated to dryness at 70-80° C. The resulting product was further purified by recrystallization from the combination of 5 ml of water and 75 ml of isopropanol, giving the desired product. The desired product consists of 88% ergothioneine (Compound V).

¹H NMR (400 MHZ, D₂O-d₆) δ 6.72 (S, 1H), 3.83 (dd, 1H), 3.09-3.15 (m, 11H).

Although specific embodiments and examples of this invention have been illustrated herein, it will be appreciated by those skilled in the art that any modifications and variations can be made without departing from the spirit of the invention. The examples and illustrations above are not intended to limit the scope of this invention. Any combination of embodiments of this invention, along with any obvious extension or analogs, are within the scope of this invention. Further, it is intended that this invention encompass any arrangement, which is calculated to achieve that same purpose, and all such variations and modifications as fall within the scope of the appended claims.

All the features disclosed in this specification (including any accompanying claims, abstract and drawings) may be replaced by alternative features serving the same, equivalent, or similar purpose, unless expressly stated otherwise. Thus, unless expressly stated otherwise, each feature disclosed is one example of a generic series of equivalent or similar features.

It is to be understood that while the invention has been described in conjunction with the detailed description thereof and accompanying FIGURES, the foregoing description and accompanying FIGURES are only intended to illustrate, and not limit the scope of the invention, which is defined by the scope of the appended claims. Other aspects, advantages, and modifications are within the scope of the following claims. All publications referenced herein are incorporated by reference in their entireties.

What is claimed is:

1. A process for synthesizing different optical isomers of ergothioneine, or a physiologically acceptable salt thereof, comprising the following successive steps:
(a) in a first solvent, reacting histidine [Compound (I)] with a protective reagent I and a protective agent II in the presence of a base, thereby obtaining a Compound (II); wherein the histidine is in L-form, D-form, or a mixture of L- and D-forms in any ratio;
(b) in a second solvent, reacting the Compound (II) with an alkylation reagent, thereby obtaining a Compound (III);
(c) in a third solvent, hydrolyzing the Compound (III) in the presence of an acid, obtaining a Compound (IV);
(d) in a fourth solvent, reacting the compound (IV) with a halogenated reagent, and then reacting with a vulcanization reagent selected from the group consisting of L-cysteine, (D,L)-cysteine, and D-cysteine, followed by reacting with a cleavage reagent, thereby obtaining ergothioneine [Compound (V)] after a post-treatment process;
wherein the obtained ergothioneine is in L-form, D-form, or a mixture of L- and D-forms in any ratio.

2. The method of claim 1, wherein the Compound (II) has the structure of Formula II, wherein R is

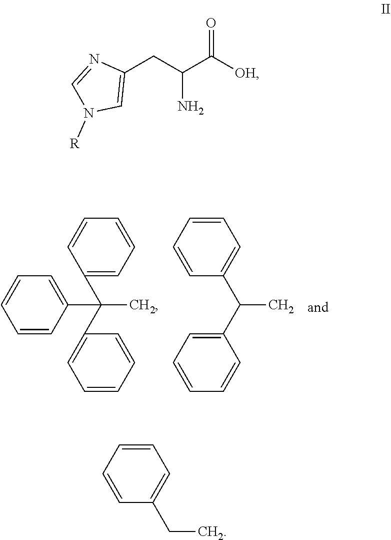

3. The method of claim 1, wherein the Compound (III) has the structure of formula III, wherein, R is

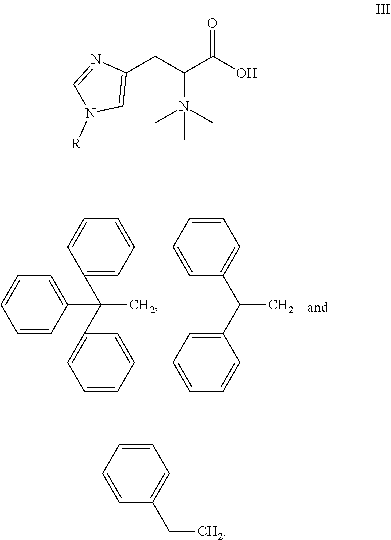

4. The method of claim 1, wherein the Compound (IV) has the structure of Formula IV,

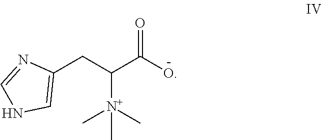

5. The method of claim 1, wherein the Compound (V) has the structure of Formula V,

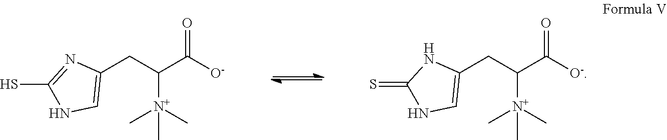

6. The method of claim 1, wherein in step (a), the protective reagent 1 is selected from the group consisting of dimethyldichlorosilane, trimethylchlorosilane, and dichlorodiphenylsilane; the protective reagent 2 is selected from the group consisting of triphenylchloromethane, chlorodiphenylmethane, and benzyl bromide; the first solvent is selected from the group consisting of methylene dichloride, tetrahydrofuran and trichloromethane; and the base is selected from the group consisting of pyridine, N,N-diisopropylethylamine and triethylamine.

7. The method of claim 6, wherein the protective reagent 1 is dimethyldichlorosilane, the protective reagent 2 is triphenylchloromethane, the first solvent is dichloromethane, and the base is triethylamine or pyridine.

8. The method of claim 1, wherein in step (a), molar ratio of the base and the compound (I) ranges from 1:1 to 3:1; wherein molar ratio of the protective reagent 1 and the Compound (I) ranges from 1:1 to 3:1, wherein molar ratio of the protective reagent 2 and the compound (I) ranges from 1:1 to 3:1, wherein the reaction temperature ranges from 0-80° C. or from 10 to 40° C.

9. The method of claim 1, wherein in step (b), the alkylation reagent is selected from the group consisting of dimethyl sulfate, methyl iodide, methyl bromide or methyl chloride; wherein the second solvent is selected from the group consisting of acetonitrile, methanol, ethanol or water.

10. The method of claim 9, wherein the alkylation reagent is dimethyl sulfate, and the second solvent is methanol.

11. The method of claim 1, wherein in step (b), the molar ratio of the alkylation reagent and the Compound (II) ranges from 1:1 to 3:1; wherein the reaction temperature ranges from 20 to 100° C. or from 20 to 30° C.

12. The method of claim 1, wherein in step (c), the third solvent is selected from the group consisting of acetonitrile, methanol, ethanol or water; the acid is selected from the group consisting of hydrochloric acid, sulfuric acid, trifluoroacetic acid, and trifluoromethanesulfonic acid.

13. The method of claim 12, wherein the third solvent is water, and the acid is hydrochloric acid.

14. The method of claim 1, wherein in step (c), the molar ratio of the acid and Compound (III) ranges from 1:1 to 10:1 or from 2:1 to 5:1; wherein the reaction temperature ranges from 20 to 80° C. or from 70 to 80° C.

15. The method of claim 1, wherein in step (d), the fourth solvent is selected from the group consisting of water, methanol and ethanol; the halogenated reagent is selected from the group consisting of bromine, dibromohydantoin, imidazolidinedione, bromosuccinimide, iodosuccinimide, chlorosuccinimide; and the cleavage reagent is selected from the group consisting of cysteamine, sodium thiosulfate, ammonium thiocyanate, and mercaptopropionic acid.

16. The method of claim 15, wherein the fourth solvent is water, the halogenated reagent is bromosuccinimide, and the cleavage reagent is sodium thiosulfate.

17. The method of claim 1, wherein in step (d), the molar ratio of the vulcanization reagent and the Compound (IV) ranges from 1:1 to 10:1 or from 3:1 to 5:1.

18. The method of claim 1, wherein in step (d), the molar ratio of the cleavage reagent and Compound (IV) ranges from 1:1 to 5:1 or from 2:1 to 3:1; and the reaction temperature is selected from 0 to 100° C. or from 70 to 90° C.

19. The method of claim 1, wherein in step (d), the post-treatment process comprises at least one step selected from the group consisting of filtration, decolorization, electrodialysis, concentration, ion-exchange chromatography and recrystallization in a recrystallization reagent.

20. The method of claim 19, wherein the recrystallization reagent is at least one reagent selected from the group consisting of isopropanol, ethanol, methanol, and water.
